(19) United States
(12) Patent Application Publication
(10) Pub. No.: US 2026/0306387 A1
(43) Pub. Date: Oct. 8, 2026

(54) Promotion of Hair Growth

(71) Applicant: CarboCode S.A., Cantanhede (PT)

(72) Inventors: Cornelia Pyko, Cantanhede (PT); Feryel Adam, Cantanhede (PT)

(21) Appl. No.: 19/483,511

(22) PCT Filed: May 9, 2024
(86) PCT No.: PCT/IB24/54527

§ 371 Date: Nov. 11, 2025

(30) Foreign Application Priority Data
May 12, 2023 (PT) 118648

(51) Int. Cl. A61K 8/68; A61Q 7/00

(52) U.S. Cl. CPC A61K 8/68; A61Q 7/00; A61K 2800/78

(57) ABSTRACT

The present invention relates to a use of a composition comprising a compound of formula I, wherein R¹ is an alkyl chain having 10-20 carbon atoms, R² is an acyl having 16-34 carbon atoms, X is hydrogen, a glucosyl moiety or a lactosyl moiety for promoting hair growth or for preventing hair loss on skin of a subject.

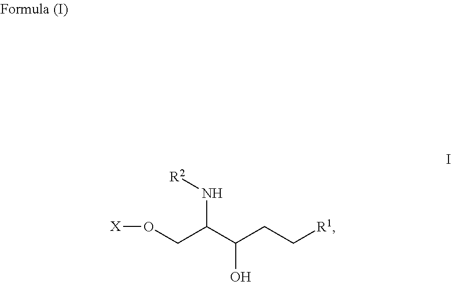

### FIELD OF THE INVENTION

The present invention relates to a use of a composition for promoting hair growth or for preventing hair loss on skin of a subject.

### BACKGROUND OF THE INVENTION

Hair is a filamentous biomaterial, which grows on human scalp approximately 0.3 mm/day, while it sheds 100 hairs/day. Hair often refers to two structures: the part beneath the skin, i.e. the hair bulb, and the hair shaft which is the hard filamentous part extending above the skin surface. Hair affects the impacts of thermoregulation, physical protection and social interaction. In humans, the main function of hair is an important facet of appearance. Hair loss or alopecia is defined as the loss of hair from an area of the body. Hair loss or slowed hair growth affects millions of people, including over 40% of men over the age of 30 and a significant number of women also. Factors responsible for hair loss include scarring, disease, infection, less blood circulation in the blood capillaries of scalp and androgen sensitivity. Although alopecia is a non-life-threatening condition, it can affect the aesthetics, social activities, and quality of life of individuals. Hair growth is the result of the growth and differentiation of hair follicles comprising dermal papilla cells and epithelial cells. Hair follicle stem cells sustain growth and cycling of the hair follicle and are located in the permanent portion of the follicle known as the bulge. Cyclical changes in hair follicle growth are divided into different stages, referred to as anagen (growth), catagen (regression), telogen (rest), and exogen (shedding). The human hair cycle transition is controlled by several growth stimulatory or inhibitory factors. During anagen, rapidly proliferating progenitor cells in the bulb generate the hair and its surrounding inner root sheath. The onset of catagen is marked by cessation of proliferation and apoptosis of the epithelial cells below the bulge. The mesenchymally derived dermal papilla survives catagen and moves upward to abut the lowermost portion of the bulge, which then forms the secondary germ at its base, during telogen. Ill-defined mesenchymal-epithelial interactions likely involving BMP and WNT signaling are thought to signal anagen onset and the generation of the new lower follicle and hair shaft. As the new hair grows in, the old hair is shed during exogen. The duration of each stage varies depending on the type, site, and genetic programming of the follicle. Often, the growth phases of the hair shorten with increasing age, leading to the formation of shorter and/or finer hair. Shifting the ratio of actively growing hair to hair in the resting phase can lead to reduced hair density. In the extreme case, hair growth cycle comes to a complete stop, resulting in baldness.

Drug-based therapies for the treatment of hair loss are available, but they have shown various side effects in patients. Thus, the use of new approaches using bioactive products to promote hair growth have been emphasized. In the cosmetic industry, a number of compounds have been described in order to reduce the visible effects of alopecia. Some substances can prevent or reduce the loss of hair. Other substances can promote hair growth, particularly by promoting the hair follicles and as a result initiating a new hair growth cycle.

There remains a need for cosmetic ingredients having few or no side effects that promote hair growth or prevent hair loss in a subject.

### SUMMARY OF THE INVENTION

The present inventors have surprisingly found that ceramide NG efficiently promotes hair growth or reduces hair loss on skin of a subject. Ceramide NG is especially targeted at extending the anagen growth phase of the hair cycle and activating the hair follicle stem cells.

The present invention comprises the following aspects:
(1) Use of a composition comprising a compound of formula I

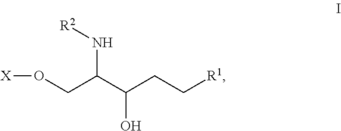

wherein
R¹ is an alkyl chain having 10-20 carbon atoms,
R² is an acyl having 16-34 carbon atoms,
X is hydrogen, a glucosyl moiety or a lactosyl moiety,
for promoting hair growth or for preventing hair loss on skin of a subject.
(2) Method for promoting hair growth or preventing hair loss on skin of a subject, the method comprising administering to the skin a composition comprising a compound of formula I.
(3) Method for activating a hair cycle on skin of a subject, the method comprising administering to the skin a composition comprising a compound of formula I.

In particular, the composition is a topical composition and used for cosmetic purposes on the scalp and/or the hair of a human, i.e. a cosmetic hair composition, or on the hair or fur of a domestic animal.

Various non-limiting embodiments relating to the above aspects of the invention are described throughout specification of the invention and illustrated by non-limiting examples.

### FIGURES

FIG. 1 shows a heatmap using a log 2 scale with upregulation (shown in black) or downregulation (shown in white) of respective proteins upon treatment with 0.01% (01), 0.03% (03) and 0.15% (15) of ceramide NG in vehicle as compared to vehicle without ceramide (VEH); detailed experimental setup explained in Example 1.

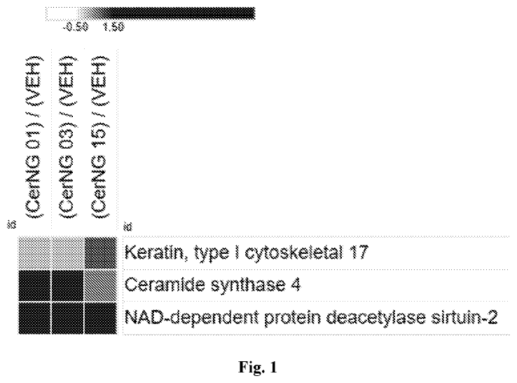

### DETAILED DESCRIPTION OF THE INVENTION

It has now been surprisingly found that compounds of formula I promote hair growth. In one aspect, the present invention relates to a use of a composition for promoting hair growth or for preventing hair loss on skin of a subject, wherein the composition comprises a compound of formula I

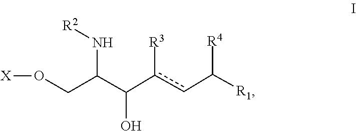

wherein
R¹ is an alkyl chain having 10-20 carbon atoms,
R² is an acyl chain having 16-34 carbon atoms,
X is hydrogen, a glucosyl moiety or a lactosyl moiety.

As used herein, the term “alkyl” refers to an acyclic straight hydrocarbyl group in which the carbon atoms may be saturated or contain one or more double and/or triple bonds (so, forming for example an alkenyl or an alkynyl). Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl. Typically, the term alkyl refers to a saturated hydrocarbyl group.

The composition of the present invention is intended for topical application. A “topical composition” as referred to herein includes compositions suitable for topical application on keratinous tissue. Keratinous tissue includes keratin-containing layers disposed as the outermost protective covering of mammals and includes herein especially skin, scalp and hair. Topical compositions are typically dermatologically acceptable in that they do not have undue toxicity, incompatibility, instability, allergic response, and the like, when applied to skin.

The composition of the present invention is especially intended for cosmetic purposes on the scalp and scalp hair of a human and is preferably a cosmetic hair composition, or on the hair or fur of a domestic animal such as a dog, a cat or a horse.

“Hair” is defined as a stratified squamous keratinized epithelium made of multi-layered flat cells whose rope-like filaments provide structure and strength to the hair shaft. Hair as used herein refers to both hair on the scalp of a human or to hair on the body of an animal, where it can be used herein interchangeably with “fur”.

The skilled person will understand that the “subject” according to the present invention refers to a mammal having hair.

In some preferred embodiment, the subject is a human. The human is in some embodiments a man. Men are more often affected by hair loss compared to women. They often suffer from a gender-specific form of hair loss, so-called androgenetic or androgenic alopecia. This form of hair loss is hereditary and often arises at young age. The main cause of this form of hair loss is the steroid hormone dihydrotestosterone (DHT). It is formed in the human body by the enzyme steroid-5-alpha-reductase from the male sex hormone testosterone and, in the event of inherited DHT hypersensitivity, leads to the shortening of the anagen phase of the hair. Another form of hair loss, alopecia areata, refers to locally limited, circular hair loss. This form of hair loss is an inflammatory autoimmune disease caused by lymphocytes that attack the bulb of hair follicles in the anagen phase, and it can occur at any stage.

The human is in other embodiments a woman. One form of hair loss which is primarily observed in women is diffuse alopecia. Diffuse alopecia manifests in a time-limited loss of hair, the possible causes being diverse, e.g. hormone fluctuations, stress or lack of iron.

Factors causing hair loss in humans include especially a reduction in hair follicle function due to male hormones, reduction in metabolic functions of hair follicles and hair bulbs, reduction in scalp physiological functions and local impairment of the circulation due to tension in the scalp.

In some preferred embodiment, the subject is a domestic animal. Included are e.g. dogs, cats, horses, sheep, rabbits and hamsters. Preferred domestic animals include dogs, cats and horses. While hair of domestic animals basically has the same hair cycle stages as in humans, there are still several differences between human hair and of domestic animals. For example, humans grow singular hairs which continuously grow, whereas dog's fur grows in bundles within each follicle, growing and shedding in cycles that differ by breed and depend on season and temperature. These bundles consist of a primary outer coat which can be coarse and a secondary soft undercoat. Moreover, the core of the hair follicle allows for the coating of hair to provide insulation from heat, cold and rain.

Hair of domestic animals can also suffer from different forms of alopecia. Alopecia may e.g. show as bald circles on parts of the animal fur. Domestic animals may acquire alopecia e.g. from a parasitic infestation of fleas, lice, mosquitoes, inadequate diet, food allergies, fungal or bacterial infections. Endocrine diseases such as hypothyroidism, cushing's disease, or reactions to rabies and corticosteroid injections can also cause alopecia. Other forms of alopecia known in dogs include e.g. seasonal flank alopecia, showing up during the cold winter months on both sides of the abdomen in front of the rear legs, or alopecia X.

“Treatment” or any variation of the term such as treating means to address a condition with the objective of improving or stabilising an outcome in the subject being treated or addressing an underlying need. Treating therefore includes the management of the condition or dermatological needs of the subject being treated. Treating can include cosmetic or medical treatment. Treatment can include preventive or curative treatment. Preventive treatment can include primary prevention or secondary prevention.

The compound of formula I may be obtained by any technique known to the skilled person. The sphingoid base moiety may e.g. be produced by a fermentation process, such as e.g. described in Schaffer, S. et al; Metabolic Engineering (2012) 14:412-426, or by a chemical process, such as e.g. described in WO 00/68238 or in He, Linli et al; Journal of Organic Chemistry (2000) 65 (22): 7618-7626. The sphingoid base may then be acylated by any known technique, e.g. enzymatically by the use of lipases, or chemically as e.g. described in WO 2013038985 or in Drug. Del. Sci. Tech. 2014, 24, 689-693, Org. Process Res. Dev. 2019, 23, 452-461.

In preferred embodiments, R¹ of the compound of formula I is —C₁₂H₂₅.

In preferred embodiments, R² of the compound of formula I is an unsubstituted acyl group. More preferably, R² of the compound of formula I is a saturated fatty acyl group or a fatty acyl group having one or more double bonds, including especially hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl and 30-(linoleyloxy)-triacontanoyl. Even more preferably, R² of the compound of formula I is an unsubstituted acyl group having 18-26 carbon atoms

In some preferred embodiments, R¹ of the compound of formula I is —C₁₂H₂₅ and R² is octadecanoyl.

In preferred embodiments, X of the compound of formula I is hydrogen.

In the context of the invention it has been found that application of ceramide NG, also termed herein as Cer NG or CNG, on keratinocytes leads to an up-regulation of proteins associated with the hair cycle.

Ceramides as used herein are named according to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. For each species, the number of carbons and unsaturations (if present) may be expressed in parentheses following the letters of N, A, E, and O. When not specified, the carbon chain length of the sphingoid base moiety of the ceramides mentioned herein is typically C₁₈. When not specified, the carbon chain length of the acyl moiety of the ceramides mentioned herein may be e.g. C₁₆, C₁₇, C₁₈, C₁₉, C₂₀, C₂₂, C₂₄, C₂₆, C₂₈, C₃₀, C₃₂ or C₃₄.

Accordingly, Cer NG refers to a ceramide comprising a non-hydroxy acid as acyl moiety and dihydrosphingosine as sphingoid base moiety. Typically, both carbon chain lengths are C₁₈. Ceramide NG may also be referred to as ceramide NdS and can be used herein interchangeably. Dihydrosphingosine may also be referred to as sphinganine and can be used herein interchangeably.

Generally, the compositions of the present invention will contain from about 0.0001% to about 10% wt/wt of a compound of formula I, such as from about 0.005% to about 5% wt/wt of a compound of formula I, or from 0.05% to about 0.2% wt/wt, from 0.01% to about 1% wt/wt of a compound of formula I. For example, the topical compositions of the present invention may contain from about 0.0001 to about 0.00025% wt/wt of a compound of formula I, from about 0.0002 to about 0.0004% wt/wt of a compound of formula I, from about 0.0003 to about 0.0005% wt/wt of a compound of formula I, from about 0.0004 to about 0.0006% wt/wt of a compound of formula I, from about 0.0005 to about 0.0008% wt/wt of a compound of formula I, from about 0.0007 to about 0.001% wt/wt of a compound of formula I, from about 0.001 to about 0.005% wt/wt of a compound of formula I, from about 0.003 to about 0.008% wt/wt of a compound of formula I, from about 0.005 to about 0.01% wt/wt of a compound of formula I, from about 0.008 to about 0.05% wt/wt of a compound of formula I, from about 0.05 to about 0.08% wt/wt of a compound of formula I, from about 0.06 to about 0.1% wt/wt of a compound of formula I, from about 0.08 to about 0.1% wt/wt of a compound of formula I, etc.

In some embodiments, topical compositions of the present invention contain an amount of about 0.01% to 5% wt/wt of a compound of formula I, or an amount of about 0.02% wt/wt to 1% wt/wt of a compound of formula I. Examples are 0.05% wt/wt, 0.1% wt/wt or 0.2% wt/wt of a compound of formula I.

The terms “about” or “approximately” are defined as being close to the value or range following these terms, as understood by one of ordinary skill in the art, and include a deviation of up to 10% of the values or ranges that follow.

The composition may in some embodiments comprise more than one compound of formula I, also termed herein as blend of compounds of formula I. In such blend compositions, each compound of formula I may be present in the same amount or in different amounts. The ranges or amounts as listed above may relate to the total content of the blend in the composition, or to the amount of one compound in the blend in the composition.

The composition according to the present invention may also include additional ingredients typically found in hair care formulations and may be selected with regard to their function. Examples include surfactants, including cationic, anionic, amphoteric, zwitterionic and ionic surfactants, etc., solvents like water or alcohols, solubilizers like ethanol, ethylene glycol, propylene glycol, etc., moisture retainers like glycerin, trehalose, sorbitol, maltitol, dipropylene glycol, 1,3-butylene glycol, sodium hyaluronate, etc., antioxidants like tocopherol, BHT, etc., UV absorbants like benzophenone derivatives, paraamino benzoate derivatives, methoxy cinnamate derivatives, etc., UV-scattering agents like inorganic compounds such as zinc oxide, zirconium oxide, titanium oxide, etc., thickeners, metal chelating agents like edetic acid salts, etc., bactericides, preserving agents like phenoxyethanol, formaldehyde solution, parabens, pentanediol or sorbic acid, emollients, vitamins, anti-inflammatory agents and anti-dandruff agents such as zinc pyrithione, selenium sulfide, salicylic acid, coal tar, sulfur, ketoconazole and climbazole, pH control agents such as citric acid, lactic acid, malic acid, glycolic acid, etc.

The composition of the present invention may be formulated as a leave-on or a rinse-off composition, preferably as a rinse-off composition. “Leave-on compositions” refer to compositions to be applied, typically after hair wash, and allowed to remain on the keratinous tissue, such as the hair and the scalp. These leave-on compositions are to be distinguished from rinse-off compositions, which are applied to the hair or the scalp/skin, either before or after the washing, and subsequently (typically a few minutes) removed either by washing, rinsing, wiping, or the like. Non-limiting examples for rinse-off formulations include shampoo, conditioner, pre- or post-shampooing serum. Non-limiting examples for leave-on formulations include hair tonic, foam or cream.

The formulation may be any type of formulation, such as a solid, semi-solid, liquid or emulsion. It may also depend on the subject that it is intended to be applied on. For example, for humans or dogs liquid shampoos are typical, whereas horses or cats may rather be treated with semi-solid or emulsion formulations, like cream formulations.

Especially when the cosmetic compositions according to the invention are hair cleansing compositions such as shampoos, they comprise additionally surfactants such as anionic, non-ionic, cationic or amphoteric surfactants.

The anionic surfactant may include C8-C24 alkyl sulfate, C8-C24 alkyl ether sulfate, CBC&4 alkyl benzene sulfonate, C8-C&& alkyl phosphate, C8-C&4 polyoxyalkylenealkyl ether phosphate, C8-C24 alkyl sulfosuccinate, C8-C24 polyoxyalkylenealkyl ether sulfosuccinate, Cs-Cz4 acyl alaninate, C8-C,,acyl N-methyl-[beta]-alaninate, C~-C74 acyl glutamate, C8-C&4 acyl isethionate, C8-C&4 acyl sarcosinate, C8-C,, taurinate, C8-C&4 acyl me10 thyl taurinate, [alpha]-sulfofatty acid ester salt, ether carbonate, polyoxyalkylene fatty acid monoethanol amide sulfonate or C8-C24 long chain carboxylate.)

The nonionic surfactant may include alkanol amide, glycerin fatty acid ester, polyoxyl5 alkylenealkyl ether, polyoxyalkyleneglycol ether, polyoxyalkylenesorbitan fatty acid ester, sorbitan fatty acid ester, polyoxyalkylenesorbit fatty acid ester, sorbit fatty acid ester, polyoxyalkyleneglycerin fatty acid ester, polyoxyalkylene fatty acid ester, polyoxyalkylenealkylphenyl tetrapolyoxyalkylene ethylenediamine-condensed substances, sucrose fatty acid ester, polyoxyalkylene fatty acid amide, polyoxy20 alkyleneglycol fatty acid ester, polyoxyalkylene castor oil denvatives, polyoxyalkylenehardened castor oil derivatives, or alkylpolyglycoside, polyglycerin fatty acid ester.

The amphoteric surfactant may include C8-C24 alkyl amidopropylbetaine, C8-C24 alkyl carboxybetaine, C8-C~4 alkyl sulfobetaine, C8-C~4 alkyl sulfobetaine, C8-C~4 alkyl hy25 droxysulfobetaine, C8-C24 alkyl amidopropylhydroxysulfobetaine, C8-C24 alkyl hydroxyphosphobetaine, C8-C&4 alkyl aminocarboxylate, C8-C24 alkyl imidazoliumbetaine, C8-C74 alkyl amineoxide, C8-C24 alkyl phosphate or esters containing a tertiary or quaternary nitrogen group, wherein C,-C,means having any number from 8 to 24 carbon atoms and being linear or branched.

The cationic surfactants may include alkyltrimethyl ammonium salts, dialkyldimethyl ammonium salts, alkylpyridium salts, alkyldimethylbenzyl ammonium salts, benzethonium chloride or benzalkonium chloride.

Especially when the cosmetic compositions according to the invention are hair conditioning compositions such as hair conditioners, they may comprise additionally further conditioning agents.

The conditioning effect may be enhanced e.g. by adding silicone (methylpolysiloxane, 10 methylphenylsiloxane, high polymeric methylpolysiloxane, cyclic polysiloxane, polyether-modified silicone, amino-modified silicone, etd.) to a hair treatment composition or skin care composition of the present invention. The amount of a silicone added is preferably 5% by weight or lower with respect to the total weight of the entire composition.

In some embodiments, the composition may advantageously comprise one or more further active ingredients for promoting hair growth or for preventing hair loss. Included are e.g. various vitamins, in particular vitamins of the B series, e.g. vitamin B7 or biotin, amino acids, in particular L-valine or L-arginine, and amino acid derivatives, e.g. creatine or L-carnitine, which, inter alia, stimulate the cell metabolism, caffeine which stimulates blood flow through the scalp and indirectly hair growth, or a plant extract.

In some embodiments, the composition may comprise one or more further active ingredients for improving hair and scalp care and/or for strengthening damaged hair.

The composition according to the present invention preferably comprises at least one cosmetically acceptable carrier. The expression “cosmetically acceptable” means a composition or an agent, which is non-toxic and physiologically acceptable and has optionally further a pleasant appearance, odour, feel and/or taste. Preferably, the carrier component is selected from water, water-miscible organic solvents, preferably C2-C4-alkanols, in particular ethanol, oils, fats, waxes, esters of C6-C30-monocarboxylic acids with mono-, di- or trihydric alcohols, saturated acyclic and cyclic hydrocarbons, fatty acids, fatty alcohols, propellant gases and mixtures thereof.

The cosmetic compositions of the present invention may further comprise substances generally known to be comprised in cosmetic compositions for their multifunctional properties with regard to cosmetic and/or formulating effects, such as amino acids (alginin, glutamic acid, etc.), higher alcohols, higher fatty acids like lauric acid, myristic acid, palmitic acid, stearic acid, behenic acid, isostearic acid, oleic acid, undecylenic acid, tall oil fatty acid, coconut oil fatty acid, palm oil fatty acid, palm kernel fatty acid, linolic acid, linoleic acid, eicosapentaenoic acid, docosahexanoic acid, etc. and esters thereof like hexyl laurate, isopropyl myristate, octyldodecyl myristate, myristic acid myristate, myristic acid-2-hexyldecyl, glycerin trimyristate, isopropyl palmitate, palmitic acid-2-heptylundecyl, palmitic acid-2-hexyldecyl, butyl stearate, isocetyl stearate, 12-hydroxystearic acid cholesteryl, cetostearylalcohol, cetyl octanate, hexyldecyldimethyl octanate, isocetyl isostearate, trimethylolpropane triisostearate, decyl oleate, oleic acid oil, cetyl lactate, myristyl lactate, ethyl acetate, butyl acetate/amyl acetate, acetic acid lanolin, 2-ethylhexanoic acid cetyl, 2-ethylhexyl palmitate, di-2-ethylhexylic acid ethylene glycol, tri-2-ethylhexylic acid trimethylol propane, tri-2-ethylhexylic acid glycerin, tetra-2-ethylhexylic acid pentaerythritol, cetyl-2-ethyl hexanoate, diisobutyl adipate, adipic acid-2-heptylundecyl, adipic acid-2-hexyldecyl, dipentaerythritol fatty acid ester, neopentyl glycol dicaprylate, diisostearyl malate, di-2-heptylundecanoic acid glycerin, tri-2-heptylundecanoic acid glyceride, castor oil fatty acid methyl ester, acetoglyceride, N-lauryl-L-glutamic acid-2-octyldodecyl ester, di-2-ethylhexyl sebacate, diisopropyl sebacate, succinic acid-2-ethylhexyl, triethyl citrate, ethyl laurate, mink oil fatty acid ester, etc.) as well as waxes like carnauba wax, candelilla wax, etc., hydrocarbon oils like liquid paraffin, squalane, etc. or essential oils. Further examples of suitable additional components can be found in the other references cited herein as well in the International Cosmetic Ingredient Dictionary and Handbook.

As typical for cosmetic products, perfumes and fragrances may be added, the smell or odour of the cosmetic formulation to be applied matters. Perfume oils which may be mentioned are mixtures of natural and synthetic fragrances. Natural fragrances are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, oranges), roots (mace, angelica, celery, cardamom, costus, iris, calmus), woods (pinewood, sandalwood, guaiacwood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic fragrance compounds are products of the ester, ether, aldehyde, ketone, alcohol and hydrocarbon types. Fragrance compounds of the ester type are e.g. benzyl acetate, phenoxyethyl isobutyrate, p-tert-butyl cyclohexyl acetate, linalyl acetate, dimethylbenzyl carbinyl acetate, phenyl ethyl acetate, linalyl benzoate, benzyl formate, ethylmethyl phenyl glycinate, allyl cyclohexyl propionate, styrallyl propionate and benzyl salicylate. The ethers include, for example, benzyl ethyl ether; the aldehydes include e.g. the linear alkanals having 8 to 18 carbon atoms, citral, citronellal, citronellyloxyacetaldehyde, cyclamenaldehyde, hydroxycitronellal, lilial and bourgeonal, the ketones include e.g. the ionones, isomethylionone and methyl cedryl ketone, the alcohols include anethole, citronellol, eugenol, isoeugenol, geraniol, linalool, phenylethyl alcohol and terpineol, the hydrocarbons include primarily the terpenes and balsams. However, preference is given to using mixtures of different fragrances which together produce a pleasant scent note. Essential oils of relatively low volatility, which are mostly used as aroma components, are also suitable as perfume oils, e.g. sage oil, chamomile oil, clove oil, melissa oil, mint oil, cinnamon leaf oil, linden blossom oil, juniper berry oil, vetiver oil, olibanum oil, galbanum oil, labolanum oil and lavandine oil. Preferably, bergamot oil, dihydromyrcenol, lilial, lyral, citronellol, phenylethyl alcohol, hexylzimtaldehyde, geraniol, benzylacetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil,-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillate, irotyl and floramat, alone or in mixtures, are used. Suitable aromas are, for example, peppermint oil, spearmint oil, anise oils, star anise oil, caraway oil, eucalyptus oil, fennel oil, lemon oil, wintergreen oil, clove oil, menthol and the like.

In the context of the invention it has been found that application of Cer NG on keratinocytes leads to an up-regulation of biomarkers associated with the hair cycle. More precisely, the application of Cer NG leads to up-regulation of ceramide synthase 4, keratin type I cytoskeletal 17 (also referred to herein as Keratin 17) and NAD-dependent protein deacetylase sirtuin-2 (also being referred to herein as Sirtuin-2).

In some embodiments, Cer NS is used for up-regulating NAD-dependent protein deacetylase sirtuin-2.

In some embodiments, Cer NG is used for up-regulating ceramide synthase 4.

In some embodiments, Cer NS is used for up-regulating keratin type I cytoskeletal 17.

The hair follows a specific growth cycle in which the anagen phase represents the period of active growth, the longer the anagen phase is the longer and thicker the hair. If the cycle is shortened, which is the case during ageing and stress hair becomes thinner. If premature hair enters the telogen phase it can cause hair loss. Hair thinning and loss are generally attributable to human follicle stem cell reservoir depletion, functional decline, and extended dormancy. Numerous transcription factors, including Sox9, Tbx1, Lhx2, TCF3/4, Foxc1 and Nfatc1, help maintain a quiescent and undifferentiated human follicle stem cell status. NFATc1 is essential for human follicle stem cell quiescence. During aging, hyper-accumulation of nuclear NFATc1 extends the dormant period of human follicle stem cells. Sirtuin-2 deacetylates Nfatc1, leading to its degradation and thus to the activation of human follicle stem cells.

Keratin 17 triggers TNF-alpha which is known to activate the hair cycle.

Ceramide synthase 4 plays an important role in the maintenance of epidermal stem cell homeostasis and regulation of the hair cycle by regulating bone morphogenetic protein (BMP) and WNT signaling.

Without being bound to theory, it is therefore believed that Cer NG can activate the hair cycle and therefore promote hair growth.

The inventive use of the composition in general is intended a subject who is a mammal. Especially, the subject is a human. In preferred embodiments, the human subject is an adult subject, e.g. a human of 20+ years old, especially 30+ years old, such as 40+ years old or 50+ years old.

The subject may in some embodiments have an exposure to stress. The exposure may be before the use of the composition of the invention, during the use of the composition of the invention and/or in the planned future relative to the use of the composition of the invention. The skilled person will understand that the term exposure typically relates to a prolonged exposure, such as for a year or many years.

The stress may be a psychological stress or an environmental stress.

Environmental stresses include solar radiation, especially ultraviolet (UV) light, polycyclic aromatic hydrocarbons (PAHs), volatile organic compounds (VOCs), ozone, nitrogen and sulphur oxides, particulate matter PM2.5, blue light, ozone, or tobacco smoke. Particulate matter (PM) is divided according to particle size: PM10 (coarse particles), PM2.5 (fine particles) and PM0.1 (ultra-fine particles), with a respective diameter of below 10, 2.5 and 0.1 μm.

The composition of the present invention may be advantageously administered to a subject for at least 7 days, such as for at least 14 days or for at least 21 days. For example, the composition is administered for at least 28 days. In practice, there is no time limitation for the administration period of a cosmetic composition of the invention.

The composition of the present invention may be administered one time or several times per day, such as 2-3 times per day. Typically, the composition is administered for consecutive days. Different aspects of the invention discussed above are described in the following selected, but not limiting, embodiments:
(1) Use of a composition comprising a compound of formula I

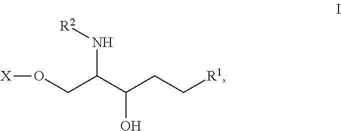

wherein
R¹ is an alkyl chain having 10-20 carbon atoms,
R² is an acyl having 16-34 carbon atoms,
X is hydrogen, a glucosyl moiety or a lactosyl moiety, for promoting hair growth or for preventing hair loss on skin of a subject.
(2) Use according to embodiment 1, wherein R¹ of the compound of formula I is-C₁₃H₂₇ and/or R² is an acyl having 18-26 carbon atoms.
(3) Use according to embodiment 1 or 2, wherein the compound of formula I is present in an amount of 0.0001% to 10% wt/wt, such as from 0.005% to 5% wt/wt, or from 0.05% to 0.2% wt/wt.
(4) Use according to any one of embodiments 1 to 3, comprising an increase of expression levels of one or more biomarkers associated with the subject's hair cycle, preferably ceramide synthase 4, keratin type I cytoskeletal 17 and/or NAD-dependent protein deacetylase sirtuin-2.
(5) Use according to any one of embodiments 1 to 4, wherein promoting hair growth or preventing hair loss on skin of a subject comprises activation of the subject's hair cycle, extension of anagen growth phase of the hair cycle and/or activation of hair follicle stem cells of the subject.
(6) Use according to any one of embodiments 1 to 5, wherein the composition further comprises a ceramide, such as ceramide NP or ceramide NS, a glycosylated ceramide, such as lactosyl ceramide or glucosyl ceramide, a sphingolipid, such as sphinganine, phytosphingosine or sphingosine, or a combination thereof.
(7) Use according to any one of embodiments 1 to 6, wherein the composition comprises a further agent for promoting hair growth or for preventing hair loss, such as gingko biloba, aloe, asiasari, proanthocyanidin, arginine, castor oil, minoxidil, taurine, caffeine, carnitine, azelaic acid, hydrolyzed keratin, biotin, creatine, niacin, panthenol, ximenynic acid, arjunolic acid, vitamin B6, zinc, copper, magnesium, ginseng, or a combination thereof.
(8) Use according to any one of embodiments 1 to 7, wherein the subject is a human or a domestic animal, such as a dog, a cat or a horse.
(9) Use according to any one of embodiments 1 to 8, wherein the skin is a scalp.
(10) Use according to any one of embodiments 1 to 9, wherein the subject is an aging subject.
(11) Use according to any one of embodiments 1 to 10, wherein the subject is or has been exposed to stress, such as psychological or environmental stress.
(12) Use according to any one of embodiments 1 to 11, wherein the subject has one or more of the following:
   a hormonal imbalance resulting in hair loss,
   a disease resulting in hair loss,
   a treatment causing hair loss,
   a heredity of premature hair loss.
(13) Use according to embodiment 12, wherein the subject is a human and the hormonal imbalance resulting in hair loss is due to menopause or post-childbirth.
(14) Use according to embodiment 12, wherein the disease resulting in hair loss includes alopecia, including lichen planopilaris, discoid lupus erythematosus and alopecia areatea.
(15) Use according to embodiment 12, wherein the treatment causing hair loss includes radiation and/or chemotherapy.
(16) Use according to any one of embodiments 1 to 15, wherein the composition is administered to a subject for at least 2 days, especially for at least 7 days or for at least 14 days, such as for 28 days.
(17) Use according to any one of embodiments 1 to 16, wherein the composition is a cosmetic composition.
(18) Use according to any one of embodiments 1 to 17, wherein the composition is formulated as a shampoo, tonic, hair conditioner, pre- or post-shampooing serum, foam or cream.
(19) Method for promoting hair growth or preventing hair loss on skin of a subject, the method comprising administering to the skin a composition comprising a compound of formula I

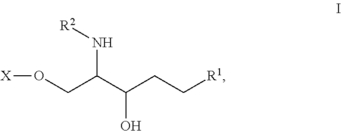

wherein
R¹ is an alkyl chain having 10-20 carbon atoms,
R² is an acyl having 16-34 carbon atoms,
X is hydrogen, a glucosyl moiety or a lactosyl moiety;
or a composition as defined in any of embodiments 1-17.
(20) Method for activating a hair cycle on skin of a subject, the method comprising administering to the skin a composition comprising a compound of formula I

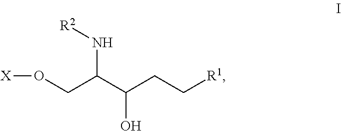

wherein
R¹ is an alkyl chain having 10-20 carbon atoms,
R² is an acyl having 16-34 carbon atoms,
X is hydrogen, a glucosyl moiety or a lactosyl moiety;
or a composition as defined in any of embodiments 1-17.

### EXAMPLES

The examples described below are for illustration purposes only. The invention is not limited in scope to embodiments described in the examples.

### Example 1: Characterisation of Effect of Ceramide NG as Cosmetic Active Ingredient

Material and methods: Reconstructed Human Epidermis (RHE) cell culture is used, as this system mimics the in vivo 3D structure of epidermal tissue as well as the conditions and processes that occur in normal epidermis. RHE are cultured using the medium provided by the manufacturer until analysis. RHE are maintained in survival for 24 hours, before RHE are topically treated with the respective ceramide in a base emulsion for 24 h. 3 RHE are treated with base emulsion alone and 3 more remained untreated as a control batch (Ctrl). RHE are incubated under classical cell culture conditions (37° C., 5% CO2).

Sample preparation and LC-MS/MS analysis: Protein is extracted using the Pierce Total Protein Extraction Kit (Thermo Fisher) according to manufacturer's recommendations. The protein is mixed with Laemmli buffer and heated to 37° C. for 30 min. Protein concentration is determined by the BCA method and standardized for all samples. The samples are separated by SDS-PAGE and digested with trypsin overnight. The peptides generated are acidified and separated. The mass spectra are queried using Proteome Discoverer (version 2.5). The resulting MS/MS data are queried against the Homo sapiens proteome UP000005640 (20371 reviewed entries). Results are filtered based on unique peptides >2, and global peptide scores. Analyses are performed using Proteome Discoverer 2.5 to check for overall sample quality.

Bioinformatics and statistical analysis: Proteomic analysis returned a mean total of 34961 high-quality peptides corresponding to a mean 2422 identifiable and quantifiable RHE proteins among conditions. Proteins with a p-value≤0.05 in at least one of the concentrations tested for a lipid are considered significant and used for pairwise comparisons. Relevant proteins are classified by their biological processes and associated pathways using the publicly available gene ontology (GO) database provided by the Gene Ontology Consortium and Reactome. Interactome analysis was performed using Cytoscape combined with STRING.

### Example 2: Effect of Cer NG on Hair Growth in Human Scalp on Skin Explants

Cer NG is topically applied to 4×6 mm scalp skin fragments at three different concentrations. One vehicle is applied as control.

The evaluated parameters include hair shaft elongation using Brightfield microscopy, skin morphology and integrity using H&E staining, hair matrix keratinocyte proliferation and apoptosis (Ki-67/Tunel immunostaining), hair cycle staging using Ki-67/Tunel immunostaining and Masson Fontana histochemistry), and number and proliferation of bulge stem cells by K15/Ki-67 immunostaining.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A method for promoting hair growth on the skin of a subject, the method comprising administering to the subject's skin a composition comprising a compound of formula I 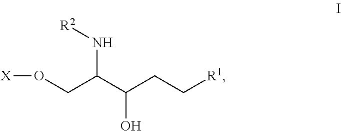
wherein
R¹ is an alkyl chain having 10-20 carbon atoms,
R² is an acyl having 16-34 carbon atoms, and
X is hydrogen, a glucosyl moiety, or a lactosyl moiety.

2. The method according to claim 1, wherein R¹ of the compound of formula I is —C₁₃H₂₇ and/or R² is an acyl having 18-26 carbon atoms.

3. The method according to claim 1, wherein the compound of formula I is present in an amount of 0,0001% to 10% wt/wt.

4. The method according to claim 1, wherein promoting hair growth comprises an increase of expression levels of one or more biomarkers associated with the subject's hair cycle.

5. The method according to claim 1, wherein promoting hair growth comprises at least one of activation of the subject's hair cycle, extension of anagen growth phase of the hair cycle, and/or activation of hair follicle stem cells of the subject.

6. The method according to claim 1, wherein the composition further comprises a ceramide, a glycosylated ceramide, a sphingolipid, or any combination thereof.

7. The method according to claim 1, wherein the composition further comprises gingko biloba, aloe, asiasari, proanthocyanidin, arginine, castor oil, minoxidil, taurine, caffeine, carnitine, azelaic acid, hydrolyzed keratin, biotin, creatine, niacin, panthenol, ximenynic acid, arjunolic acid, vitamin B6, zinc, copper, magnesium, ginseng, or any combination thereof.

8. The method according to claim 1, wherein the subject is a human or a domestic animal.

9. The method according to claim 1, wherein the skin is a scalp.

10. The method according to claim 1, wherein the subject is an aging subject.

11. The method according to claim 1, wherein the subject is or has been exposed to mental, psychological, or environmental stress, or any combination thereof.

12. Use The method according to claim 1, wherein the subject has one or more of the following:
a hormonal imbalance resulting in hair loss,
a disease resulting in hair loss,
a treatment causing hair loss, and
a heredity of premature hair loss.

13. The method according to claim 12, wherein the subject is a human and the hormonal imbalance resulting in hair loss is due to menopause or childbirth.

14. The method according to claim 12, wherein the disease resulting in hair loss is at least one of alopecia, lichen planopilaris, discoid lupus erythematosus, and alopecia areatea.

15. The method according to claim 12, wherein the treatment causing hair loss comprises radiation and/or chemotherapy.

16. The method according to claim 1, wherein the composition is administered to a subject for at least 2 days.

17. The method according to claim 1, wherein the composition is a cosmetic composition.

18. The method according to claim 1, wherein the composition is formulated as a shampoo, tonic, hair conditioner, pre- or post-shampooing serum, foam, or cream.

19. A method for reducing hair loss on the skin of a subject, the method comprising administering to the subject's skin a composition comprising a compound of formula I 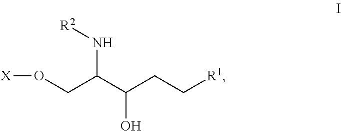
wherein
R¹ is an alkyl chain having 10-20 carbon atoms,
R² is an acyl having 16-34 carbon atoms, and
X is hydrogen, a glucosyl moiety, or a lactosyl moiety.

20. A method for activating a hair cycle on the skin of a subject, the method comprising administering to the subject's skin a composition comprising a compound of formula I 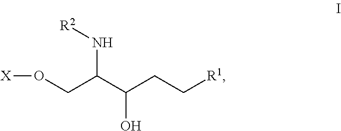
wherein
R¹ is an alkyl chain having 10-20 carbon atoms,
R² is an acyl having 16-34 carbon atoms, and
X is hydrogen, a glucosyl moiety, or a lactosyl moiety.
